(19) United States
(12) Patent Application Publication
(10) Pub. No.: US 2024/0008988 A1
(43) Pub. Date: Jan. 11, 2024

(54) ONE-WAY VALVE OPENING DEVICE, INDWELLING NEEDLE COMPRISING SAME, AND BRONCHIAL STENT

(71) Applicant: Sanming DENG, Xi'an (CN)

(72) Inventor: Sanming DENG, Xi'an (CN)

(21) Appl. No.: 18/254,337

(22) PCT Filed: May 21, 2021
(86) PCT No.: PCT/CN2021/095095

§ 371 Date: May 24, 2023

(30) Foreign Application Priority Data
Nov. 24, 2020 (CN) 202011332817.0

(51) Int. Cl. A61F 2/24; A61F 2/01

(52) U.S. Cl. CPC A61F 2/2476; A61F 2/011; A61F 2230/0067

(57) ABSTRACT

A one-way valve opening device, comprising a gasket (4) and a plurality of elastic fine fibers (3), wherein one end of each of the plurality of fine fibers (3) is fixed to the gasket (4), and the other end thereof is free; when the pressure on free ends (6) is not lower than that on fixed ends (5), the free ends (6) of the plurality of fine fibers (3) stay in a gathered state, the plurality of fine fibers (3) are in the form of a cone as a whole; and when the pressure on the free ends (6) is lower than that on the fixed ends (5), the free ends (6) of the plurality of fine fibers (3) are dispersed and are in an open state. The one-way valve opening device is placed into an indwelling needle, a thrombus (11) caused during the usage of the indwelling needle can be prevented in a needle hub (9), such that the thrombus (11) is prevented from entering a human body, and serious consequences caused by the entry of the thrombus (11) into the human body are thus reduced. The one-way valve opening device is placed in a bronchus (16) of a lesion area in a human body, such that the movement direction of sputum (19) can be limited, so that the sputum (19) is discharged out of the body, and the intake and output of gas and liquid is not limited, but same can directly reduce the air intake volume in the lung in the lesion area, indirectly improving the ventilation/perfusion blood flow ratio of a normal lung, and also does not affect subsequent treatments such as alveolar lavage.

### CROSS REFERENCE TO RELATED APPLICATION

This application claims priority to Chinese Patent Application No. 2020113328170, filed on Nov. 24, 2020, the entire content of which is incorporated herein in its entirety.

### TECHNICAL FIELD

The present disclosure relates to the technical field of medical devices, in particular, to a one-way valve open device, an indwelling needle and a bronchial stent including the same.

### BACKGROUND

Indwelling needles are conventional devices used in medical clinical application. A soft outer cannula is placed in the human blood vessel by puncture and can be kept in the body for several days, which has been widely used clinically. A common adverse reaction during the use of indwelling needles is catheter blockage. The catheter blockage of the indwelling needle is mainly caused by the thrombus formed in the soft outer cannula and the needle hub. For avoiding above-mentioned problems, at present, following methods are mainly adopted. 1, a positive pressure connector is used, such that a certain positive pressure is produced during infusion, avoiding blood reflux. However, it is impossible to completely prevent the blood from flowing back into a trocar when the infusion is not performed, thereby forming a thrombus and causing the catheter blockage, and the thrombus may enter the blood vessel of the human body during another infusion. 2. The indwelling needle is flushed every few hours when the infusion is not performed, and normal saline is injected into the patient's body through the indwelling needle to keep the indwelling needle unblocked. Flushing with normal saline may flush the thrombus in both the soft outer cannula and the needle hub into the blood vessels of the human body. Both of the above methods increases the risk of embolism in patients. A more reasonable way is to use a syringe to draw out the blood and thrombus through the indwelling needle, and then re-inject normal saline to flush the indwelling needle. Although the thrombus in the indwelling needle is drawn out in this way, the blood drawn out increases the risk of medical personnel being infected with diseases such as hepatitis B and AIDS, and increases the hospital's investment in medical waste disposal at the same time. At present, the research and development of indwelling needles is mainly focused on how to protect the safety of medical staff, avoid accidental injury to the operator by the withdrew needle, or prevent the patient's blood from flowing out of the indwelling needle to infect the operator. However, problems of how to protect the patient, how to allow the thrombus generated when the indwelling needle is indwelling in the human body to be discharged into the needle hub (outside the body) of the indwelling needle, prevent the thrombus from re-entering the body during the flushing and infusion processes, while not affecting the fluid being infused into the body smoothly through the indwelling needle has not yet been resolved.

Diseases such as bullae, emphysema, and chronic obstructive pulmonary disease are more common. Although the above diseases are benign diseases, the quality of life of patients is poor and the survival period is limited. At present, minimally invasive interventional surgery (one-way valve lung volume reduction) has been used to treat such diseases. The minimally invasive interventional surgery (one-way valve lung volume reduction) is to put a membrane-covered stent into the bronchus of the lesion area. This kind of membrane-covered stent has following characteristics: completely preventing objects (gas, liquid, and solid) from entering the lungs in the lesion area, and allow the gas and sputum in the lungs in the lesion area to be discharged from the body through the valve device, so as to achieve the effect of treating the disease. However, completely preventing the gas from passing through the one-way valve device inevitably leads to diseases such as lung collapse, atelectasis, and lung consolidation in patients, and a lot of sputum may be generated during treatment process, such that it is impossible to perform alveolar lavage (in which the normal saline is injected into the broncho-alveoli through the bronchoscope, and is then drawn out, which can promote the excretion of sputum in the deep part of the lung), thereby affecting the subsequent treatment. More reasonable approaches are to restrict the movement of sputum in one direction to prevent the accumulation of sputum in the lungs in the lesion area; to not restrict the two-way movement of gas, but to reduce the relative air intake, so as to reduce the volume of the alveoli in the lesion area and maintain a certain pressure in the alveoli, to prevent diseases such as lung collapse, atelectasis, and lung consolidation; to not restrict the two-way movement of liquid, thereby not affecting the subsequent treatment. The problem of how to design a one-way valve device for sputum to achieve the best therapeutic effect has not yet been resolved.

In view of the above-mentioned problems currently existing in the indwelling needles and endobronchial membrane-covered stents, the problem of how to design a one-way valve device for specific substances, allowing specific substances to move in one direction, and allowing other substances to move in two directions, has not yet been resolved.

### SUMMARY

To solve the above problems, the present disclosure provides a one-way valve open device. The one-way valve open device of the present disclosure includes a gasket and a plurality of fine fibers having elasticity. One end of each of the plurality of fine fibers is fixed on the gasket, and the other end of each of the plurality of fine fibers are free. When pressure at the free ends is not less than pressure at the fixed ends, the free ends of the fine fibers remain in a gathered state, and the plurality of fine fibers forms a cone as a whole. When the pressure at the free ends is less than the pressure at the fixed ends, the free ends of the fine fibers are scattered and in an open state, allowing unidirectional movement of specific substances and bidirectional movement of other substances.

The device of the present disclosure is placed in the indwelling needle, such that the thrombus generated during the use of the indwelling needle are stopped in the needle hub, preventing the thrombus from entering the human body, reducing the serious consequences caused by the thrombus entering the human body, such as pulmonary embolism, or organ embolism, reducing the blockage rate of the indwelling needle during use, while the liquid is allowed to pass in and out smoothly through the gaps between the fine fibers, without restricting the flow of the liquid.

The device of the present disclosure is placed in the bronchus in the lesion area of the human body, which can limit the movement direction of sputum, make the sputum excreted from the body, does not restrict the entry and exit of gas and liquid, but can directly reduce the air intake of the lungs in the lesion area, and indirectly improve the ratio of the ventilation to perfusion blood flow of the normal lungs, while not affecting subsequent alveolar lavage and other treatments, so as to treat pulmonary diseases such as emphysema, bullae, or chronic obstructive pneumonia.

The specific technical solution according to the present disclosure is as follows.

A one-way valve open device is provided, including a gasket and a plurality of fine fibers having elasticity. One end of each of the plurality of fine fibers is fixed on the gasket, and the other end of each of the plurality of fine fibers is free. When pressure at the free ends is not less than pressure at the fixed ends, the free ends of the plurality of fine fibers remain in a gathered state, and the plurality of fine fibers are in the shape of a cone as a whole. A puncture steel needle or guidewire can pass through the top of the cone, and after the puncture steel needle or guidewire is removed, the free ends of the fine fibers return to the gathered state. When the pressure at the free ends is less than the pressure at the fixed ends, the free ends of the fine fibers are scattered and in an open state.

Further, the gasket and the fine fibers are made of medical materials. The materials are natural materials, artificial synthetic materials, or a combination thereof. The natural materials include plants fibers and animal hair. The artificial synthetic materials include biomedical metal materials, biomedical polymer materials, and biomedical composite materials.

Further, an outline of the gasket is circular, elliptical, or polygonal, and has a cross-section being circular, elliptical, or polygonal. Further, when the device of the present disclosure is applied in the indwelling needle, the gasket is fixed at an opening of a soft outer cannula of the indwelling needle which is located at the needle hub of the indwelling needle, or fixed in the soft outer cannula, or fixed in an inner cavity of a needle hub close to the soft outer cannula. A diameter of the gasket is determined by a location of the gasket.

Further, when the device of the present disclosure is applied in the bronchus of the human body, the diameter of the gasket is determined by an inner diameter of the bronchus in which the device is placed.

Further, an included angle between the gasket and the soft outer cannula or the bronchus is preferably in a range from 80° to 100°, most preferably 90°.

Further, a shape of the fine fiber is conical, cylindrical, ellipsoid, cuboid, polyhedron, or leaf vein type, and has a cross-sectional shape being circular, oval, square, triangle, or polygon.

Further, the fine fiber can be provided with one, two or more free ends. The free ends can be fused with each other;

Further, the plurality of fine fibers can be made of the same material or different materials.

Further, the elasticity of the plurality of fine fibers can be the same or different.

Further, the number and lengths of fine fibers, an included angle between the fine fiber and the gasket, and a size of the cone formed by the plurality of fine fibers are directly determined by a diameter of the gasket. The included angles between each of the plurality of fine fibers and the gasket can be the same or different.

Further, the one end of each of the fine fibers is fixed on the gasket at a certain distance and angle. The plurality fine fibers forms one or more curved surfaces. A plurality of curved surfaces are spliced to form one or more ridge shapes, or a cone such as circular cone, triangular pyramid, or quadrangular pyramid. The cone can be formed by splicing some cones of different sizes and/or shapes, or can be formed by splicing some cones and flat or curved surfaces. In the same cone, the lengths of the above fine fibers may be equal or not. Distances between the above fine fibers distributed on the gasket may also be equal or not. The number of the fine fibers required to constitute one cone is determined by the diameter of the gasket and the diameters of the fine fibers.

Further, the free ends of the plurality of fine fibers can contact each other, not contact each other, or cross each other.

Further, the plurality of free ends can be fused to form various shapes, such as circular cone shape, fan shape, semi-circular shape, or blade shape.

Further, areas or shapes of gaps formed between the fine fibers can be the same or different.

Further, when the one-way valve open device is applied in the indwelling needle, the gasket may not be used, and the above plurality of fine fibers are directly fixed on an inner wall of the soft outer cannula of the indwelling needle or at the opening of the needle hub at a certain distance and angle.

Further, when the one-way valve open device is applied in a bronchus of the human body, the gasket can be fixed to a stent material for better fixation to a wall of the bronchus.

Optionally, an endobronchial stent is provided. A gasket is fixed on the stent. One end of the fine fibers is fixed on the gasket, and the other end of each of the fine fibers is free. When pressure at the free ends is not less than pressure at the fixed ends, the free ends of the fine fibers remain in a gathered state, and the plurality of fine fibers are in the shape of a cone as a whole. A guidewire enters a bronchus through the top of the cone. When the guidewire is removed, the free ends of the fine fibers return to the gathered state. When the pressure at the free ends is less than the pressure at the fixed ends, the free ends of the fine fibers are scattered and in an open state.

Further, when the one-way valve open device is applied in a bronchus of the human body, the gasket may not be used, and the above plurality of fine fibers are directly fixed on a stent material at a certain distance and angle. The stent includes a stent material and a plurality of fine fibers having elasticity. One end of each of the plurality of fine fibers are fixed at the stent material at a certain distance and angle, and the other end of each of the plurality of fine fibers are free. When pressure at the free ends is not less than pressure at the fixed ends, the free ends of the plurality of fine fibers remain in a gathered state, and the plurality of fine fibers are in the shape of a cone as a whole. When the pressure at the free ends is less than the pressure at the fixed ends, the free ends of the fine fibers are scattered and in an open state

Further, the number, lengths of the fine fibers, an included angle between the fine fibers and the bronchus, and a size of the cone or curved surface formed by the plurality of fine fibers are determined by a diameter of the bronchus. The included angles between the plurality of fine fibers and the bronchus can be the same or different.

An indwelling needle configured for preventing thrombus from entering human body is further provided. The indwelling needle includes a puncture steel needle, a soft outer cannula, a needle hub, a connector assembly, and the one-way valve open device as described above.

The one-way valve open device of the present disclosure can be applied to other catheters indwelled in the body, such as deep vein cannula, positive pressure connector, or PICC. In the catheters in body, one set of the device designed by the present disclosure can be used, or multiple sets of the device designed by the present disclosure can also be used.

The present disclosure further provides a device for treating pulmonary diseases, which can be used alone, or the device includes a stent and the above one-way valve open device fixed on the stent.

The present disclosure can achieve beneficial effects as follows.

The one-way valve open device of the present disclosure allows unidirectional movement of specific substances and bidirectional movement of other substances.

The device of the present disclosure is placed in the indwelling needle, such that the thrombus generated in positions of the indwelling needle, such as the soft outer cannula or the needle hub, can be stopped in the needle hub, preventing the thrombus from entering the human body, reducing the serious consequences caused by the thrombus entering the human body, such as pulmonary embolism, or organ embolism, reducing the blockage rate of the indwelling needle during use, while the liquid can pass in and out smoothly through the gaps between the fine fibers, without restricting the flow of the liquid.

When the device of the present disclosure is placed in the bronchus in the lesion area, the unidirectional movement of sputum can be limited, such that the sputum can be excreted from the body, the entry and exit of gas and liquid are not restricted, the air intake of the lungs in the lesion area are directly reduced, and the ratio of the ventilation to perfusion blood flow of the normal lungs is indirectly improved, without affecting subsequent treatment such as alveolar lavage, so as to treat lung diseases such as bronchiectasis, emphysema, or bullae.

### BRIEF DESCRIPTION OF THE DRAWINGS

FIG. 1 is a structural schematic view showing a one-way valve open device according to the present disclosure.

FIG. 2 is a schematic view showing an application of the one-way valve open device to an indwelling needle.

FIG. 2A is a schematic view showing a puncture steel needle entering a soft outer cannula through fine fibers.

FIG. 2B is a schematic view showing an indwelling needle puncturing blood vessel.

FIG. 2C is a schematic view showing the case where the puncture steel needle is removed.

FIG. 2D is a schematic view showing a removal of the puncture steel needle after the indwelling needle is punctured.

FIG. 3 is a working schematic view showing an application of the one-way valve open device to the indwelling needle.

FIG. 3A is a schematic view showing the indwelling needle indwelled in the body, where blood enters a needle hub of the indwelling needle through an indwelling needle outer cannula.

FIG. 3B shows free ends of fine fibers being scattered being in an open state when pressure at the free ends is less than that at fixed ends and when a syringe is used to perform a drawing operation, in which thrombus enters the needle hub of the indwelling needle along with blood flow.

FIG. 3C shows the free ends of the fine fibers remaining in a gathered state when the pressure at the free end is not less than that at the fixed ends and when the syringe is used to perform a pushing operation, in which the thrombus in the needle hub of the indwelling needle cannot enter the human body through the device of the present disclosure.

FIG. 3D shows the free ends of the fine fibers remaining in the gathered state when an infusion pipeline is connected and when liquid is dripped, in which the thrombus in the needle hub of the indwelling needle cannot enter the human body through the device of the present disclosure.

FIG. 4 is a schematic view showing an application of the one-way valve open device to bronchus.

FIG. 5 is a working schematic view showing the application of the one-way valve open device to the bronchus.

In figures, 1. closed state of the present disclosure patent, 2. open state of the present disclosure patent, 3. fine fiber, 4. gasket, 5. fixed end of fine fiber, 6. free end of fine fiber, 7. steel needle in indwelling needle, 8. soft outer cannula of indwelling needle, 9. needle hub of indwelling needle, 10. blood vessel, 11. thrombus, 12. GLAVE, 13. syringe, 14. infusion set, 15. guidewire, 16. bronchus, 17. end close to main bronchus, 18. end close to lung side, 19. sputum, 20. normal saline for flushing.

### DETAILED DESCRIPTION OF THE EMBODIMENTS

Technical solutions in the embodiments of the present disclosure will be clearly and completely described below the with reference to the accompanying drawings in the embodiments of the present disclosure. Apparently, the described embodiments are only some, not all, embodiments of the present disclosure. Components of the embodiments of the present disclosure generally described and illustrated in the accompanying drawings herein may be arranged and designed in a variety of different configurations. Accordingly, the following detailed description of the embodiments of the present disclosure provided in the accompanying drawings is not intended to limit the scope claimed by the present disclosure, but merely represents selected embodiments of the present disclosure. Based on the embodiments of the present disclosure, all other embodiments obtained by those skilled in the art without making creative efforts belong to the protection scope of the present disclosure.

It should be noted that like numerals and letters denote similar items in the following accompanying drawings. Therefore, once an item is defined in one figure, further definition and explanation is not required in subsequent figures. Meanwhile, in the description of the present disclosure, the terms such as “first” or “second”, are only used to distinguish descriptions, and cannot be understood as indicating or implying relative importance.

### First Embodiment

As shown in FIG. 1, a one-way valve open device of the present disclosure includes a gasket 4 and a plurality of fine fibers 3. One end of each of the fine fibers 3 is fixed and is called a fixed end 5, and the other end 6 of each of the fine fibers 3 is free. The fine fibers 3 have elasticity. When pressure at the free ends 6 is not less than pressure at the fixed ends 5, the free ends 6 of the fine fibers remain in a gathered state (as shown in FIG. 1). When the pressure at the free ends 6 is less than the pressure at the fixed ends 5, the free ends of the fine fibers 6 are scattered and in an open state (as shown in FIG. 1).

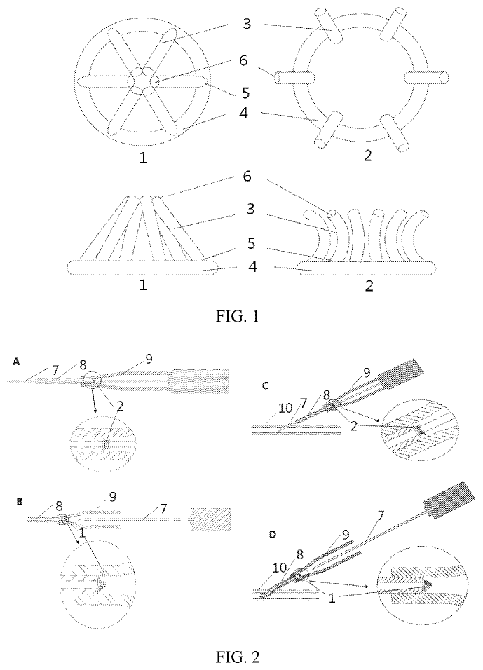

The one-way valve open device according to the present disclosure is put into an indwelling needle as a component to be used. As shown in FIG. 2A, the gasket 4 is fixed at an opening of a soft outer cannula 8 of the indwelling needle which is located at the needle hub 9 of the indwelling needle. A puncture steel needle 7 enters the soft outer cannula 8 through the free ends 6 (as shown in FIGS. 2A to 2B). After the puncture steel needle 7 is removed, the free ends 6 of the fine fibers 3 return to a closed state (as shown in FIGS. 2C to 2D).

The gasket 4 and the fine fibers 3 are made of biomedical polymer materials (such as polyether urethane, polytetrafluoroacetyl, or polyurethane). The fine fiber 3 is cylindrically shaped, and has a circular cross-sectional shape. An outline of the gasket 4 is circular. The plurality of fine fibers 3 have the same elasticity. The plurality of fine fibers 3 form a cone, the free ends of the plurality of fine fibers 3 touch each other.

The number and the lengths of fine fibers 3, and an included angle between the fine fiber and an opening of the soft outer cannula 8 is directly determined by the opening of soft outer cannula 8.

The above device is added on the basis of the needle hub 9 of the conventional indwelling needle. The indwelling needle including the device of the present disclosure is the same as the conventional indwelling needle in terms of puncture, infusion, and extubation. When the indwelling needle is kept in the body, the conventional practice is to flush the indwelling needle with normal saline every few hours to keep the indwelling needle unblocked. Compared with the conventional methods, the use of the indwelling needle including the device of the present disclosure has only one more step, and the specific method is as follows.

\textcircled{1} Some normal saline is drawn with a syringe.

\textcircled{2} The syringe is connected to a GLAVE on the back of the indwelling needle.

\textcircled{3} The syringe is used to perform a drawing operation, so that the blood enters the needle hub 9 of the indwelling needle through the outer cannula 8 of the indwelling needle (which is the only extra step).

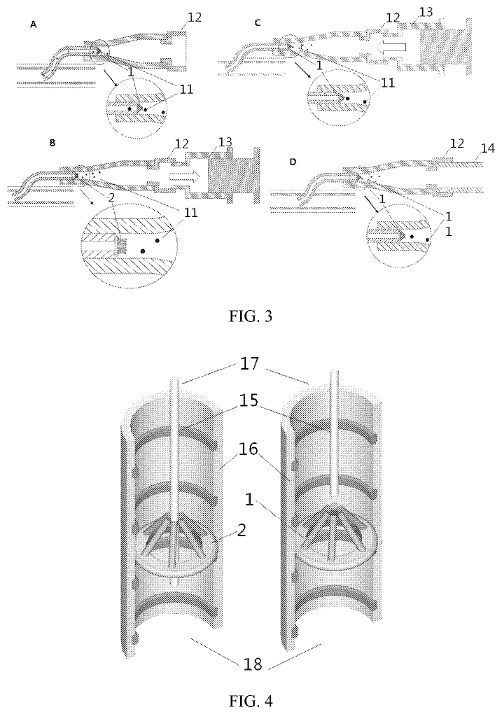

Specifically, the syringe is used to draw back the blood, thereby drawing the blood into the needle hub 9 of the indwelling needle, such that the blood enters the needle hub 9 through the soft outer cannula 8 (but the blood is avoided from being drawn out of the indwelling needle), and the fine fibers move directionally along with the liquid flow (As shown in FIGS. 3A to 3B). Therefore, an opening of the cone formed by the plurality of fine fibers 3 becomes large, such that the device is open, and the thrombus formed in the soft outer cannula 8 passes through the device along with the blood and enters the needle hub 9. In this way, the thrombus in the soft outer cannula 8 can be drawn into the needle hub 9 of the indwelling needle (as shown in FIG. 3C). When there is no liquid flow, since the fine fiber having a certain elasticity, and the opening formed at ends of the fine fibers returns to a closed state again.

\textcircled{4} The syringe is used to perfume a pushing operation, such that the normal saline passes through the GLAVE, the needle hub of the indwelling needle, and the outer cannula 8 of the indwelling needle, and then enters the blood vessels of the human body.

When the liquid enters the blood vessel through the soft outer cannula 8, the elasticity of the fine fibers and (or) the contact between the fine fibers prevent the fine fibers from moving in a direction of the liquid flow, and keep the device in a closed state (as shown in FIG. 3D). The liquid can enter the blood vessel through the gaps formed between the plurality of fine fibers, while the thrombus cannot pass through the gaps, thus blocking the thrombus in the needle hub and preventing the thrombus from entering the human body.

\textcircled{5} Sterilization and catheter lock are performed.

When re-infusion is performed through the indwelling needle, it is necessary to use the normal saline to withdraw and flush the indwelling needle again (the specific method is as described above).

### Second Embodiment

A one-way valve open device of the present disclosure can be used as a medical device, and can be put into a bronchus in a lesion area, for use in diseases such as bullae, emphysema, and chronic obstructive pulmonary disease. The structure of the one-way valve open device is similar to that of the one-way valve open device of the First Embodiment, and a cone formed by the fine fibers 3 is oriented toward a direction approaching to the main bronchus (as shown in FIG. 4).

The device as described above according to the present disclosure can be fixed on a stent material so as to be better fixed on a wall of a bronchus 16.

The present disclosure replaces the existing endobronchial membrane-covered stent, and operation steps of implantation are the same as the existing minimally invasive interventional operation. The specific method is as follows.

\textcircled{1} Under the guidance of techniques such as bronchoscopy, angiography, or chest computerized tomography (CT), a bronchial junction is found out in the lesion area, and a diameter of the bronchial junction is measured.

\textcircled{2} The device adapted to the diameter of the junction is selected, and a guidewire 15 is used to place and fix the device at the bronchial junction of the lesion area, and the guidewire 15 is withdrawn.

During exhalation, the gas is discharged from the alveoli through the trachea, such that the pressure at the free end 6 of the fine fiber 3 is less than the pressure at the fixed end 5, the fine fiber 3 moves in the direction of gas flow, and the opening of the cone formed by the plurality of fine fibers 3 becomes large, the device is open, and the gas is discharged, so that the gas output does not change much. At the end of exhalation, the pressure in the trachea is consistent, since the fine fibers 3 have a certain degree of elasticity, and the opening formed by the ends of the fine fibers 3 returns to the closed state again (as shown in FIG. 4).

During inhalation, gas enters the alveoli from the outside through the trachea, such that the pressure at the free end 6 of the fine fiber 3 is greater than the pressure at the fixed end 5. The elasticity of the fine fiber 3 itself and (or) the contact between the fine fibers prevent the fine fiber 3 from moving in the direction of the gas flow, and keep the device in the closed state. The gas can enter the bronchus 15 through the gaps formed between the plurality of fine fibers 3, thus reducing the gas intake of the lungs in the lesion area (as shown in FIG. 4).

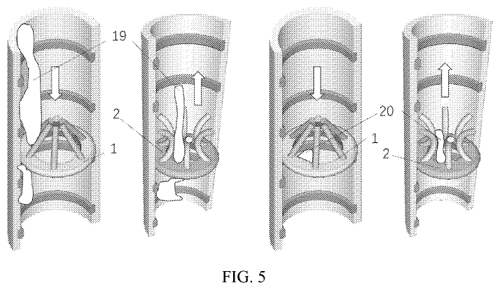

When coughing and expectoration, the pressure of the free end 6 of the fine fiber 3 is less than the pressure of the fixed end 5, and the opening of the cone formed by the plurality of fine fibers 3 becomes large, the device is open, and the sputum can be smoothly discharged through the device (as shown in FIG. 5).

In the present disclosure, the two-way flow of the liquid is not affected, and there is no difference between the subsequent operations of alveolar lavage performed on the patient and the existing related method. The specific method is as follows.

\textcircled{1} A bronchoscope is used to find a bronchial junction in the lesion area.

\textcircled{2} The normal saline is injected through the bronchoscope, full suction of the normal saline and sputum, and the bronchoscope is withdrawn.

During alveolar lavage, the liquid can enter the lungs in the lesion area through the gaps formed between the fine fibers 3. When the liquid is drawn, the pressure at the free end 6 of the fine fiber 3 is lower than the pressure at the fixed end 5, and the opening of the cone formed by the plurality of fine fibers 3 becomes large, the device is open, and the liquid and sputum can be discharged through the device smoothly (as shown in FIG. 5).

When the device is placed in the bronchus in the lesion area, the gas intake of the lungs can be directly reduced in the lesion area, the ratio of ventilation to perfusion blood flow of the normal lung can be indirectly improved, while not affecting the subsequent treatment such as alveolar lavage, so as to treat lung diseases, such as bronchiectasis, emphysema and bullae.

In the bronchus in the lesion area, one set of the device designed by the present disclosure can be used, or multiple sets of the device designed by the present disclosure can also be used.

The one-way valve open device of the present disclosure can be applied to other catheters indwelled in the body, such as deep vein cannula, positive pressure connector, or percutaneously inserted central catheter (PICC). In the intracorporeal catheter, one set of the device designed by the present disclosure can be used, or multiple sets of the device designed by the present disclosure can also be used.

It should be noted that, according to the disclosure and statement of the above specification, those skilled in the art to which the present disclosure pertains can also make changes and modifications to the above embodiments. Therefore, the present disclosure is not limited to the specific embodiments disclosed and described as above, and some equivalent modifications and changes to the present disclosure should also be fallen within the protection scope of the claims of the present disclosure. In addition, while some specific terms are used in this specification, these terms are only for convenience of description and do not constitute any limitation to the present disclosure.

It is easy for those skilled in the art to understand that the above descriptions are only preferred embodiments of the present disclosure, and are not intended to limit the present disclosure. Any modifications, equivalent replacements and improvements made within the spirit and principles of the present disclosure, should be included within the protection scope of the present disclosure.

1. A one-way valve open device, comprising:
a gasket; and
a plurality of fine fibers having elasticity,
wherein one end of each of the plurality of fine fibers is fixed on the gasket, and the other end of each of the plurality of fine fibers is free; when pressure at the free ends is not less than pressure at the fixed ends, the free ends of the plurality of fine fibers remain in a gathered state, and the plurality of fine fibers are in the shape of a cone as a whole; and when the pressure at the free ends is less than the pressure at the fixed ends, the free ends of the fine fibers are scattered and in an open state.

2. The one-way valve open device according to claim 1, wherein the gasket and the fine fibers are made of medical materials; the materials used are natural materials, artificial synthetic materials, or a combination thereof; the natural materials comprise plants fibers and animal hair; and the artificial synthetic materials comprise biomedical metal materials, biomedical polymer materials, and biomedical composite materials.

3. The one-way valve open device according to claim 1, wherein the plurality of fine fibers form a cone or part of a cone; and the cone is capable of being formed by splicing some cones of different sizes and/or shapes; the plurality fine fibers is capable of forming one or more flat surfaces or curved surfaces; a plurality of curved surfaces are spliced to form one or more ridge shapes, or the cone is capable of being formed by splicing some cones and the flat surfaces or the curved surfaces; and free tip ends of the fine fibers contact each other, do not contact each other, or cross each other.

4. The one-way valve open device according to claim 1, wherein the number and lengths of fine fibers, an included angle between the fine fiber and the gasket, and a size of the cone formed by the plurality of fine fibers are directly determined by a diameter of the gasket.

5. The one-way valve open device according to claim 1, wherein a singe fine fiber is capable of being provided with one, two or more free end portions; the free end portions are capable of forming a certain pattern; a plurality of free end portions are capable of being fused with each other; the plurality of free ends are capable of being fused to form various shapes, such as fan shape, semi-circular shape, or blade shape; and areas and shapes of gaps formed between the fine fibers are capable of being the same or different.

6. The one-way valve open device according to claim 1, wherein the gasket is capable of being closed or not closed; and a diameter of the gasket is determined by a location of the gasket.

7. An indwelling needle configured for preventing thrombus from entering human body, comprising:
a puncture steel needle;
a soft outer cannula;
a needle hub;
a connector assembly; and
the one-way valve open device according to claim 1,
wherein the indwelling needle is capable of being adapted to other catheters indwelled in a body, or an access device connected for vascular access in the body, such as an arterial catheter, deep vein cannula, PICC, or positive pressure connector.

8. The indwelling needle according to claim 7, wherein the gasket is fixed at an opening of a soft outer cannula of the indwelling needle which is located at the needle hub of the indwelling needle, or fixed in the soft outer cannula, or fixed in an inner cavity of a needle hub of the soft outer cannula; the gasket may not be used; the plurality of fine fibers are directly fixed at an inner wall of the soft outer cannula of the indwelling needle or at an opening of the needle hub of the indwelling needle at a certain interval and angle.

9. An endobronchial stent, characterized by the description of the device of the present disclosure that is fully unfolded in a bronchus, the endobronchial stent comprising the one-way valve open device according to claim 1.

10. An endobronchial stent, wherein the stent comprises:
a stent material; and
a plurality of fine fibers having elasticity;
wherein one end of each of the plurality of fine fibers is fixed at the stent material at a certain distance and angle, and the other end of each of the plurality of fine fibers is free; when pressure at the free ends is not less than pressure at the fixed ends, the free ends of the plurality of fine fibers remain in a gathered state, and the plurality of fine fibers are in the shape of a cone as a whole; and when the pressure at the free ends is less than the pressure at the fixed ends, the free ends of the fine fibers are scattered and in an open state.
